(19) United States
(12) Patent Application Publication
(10) Pub. No.: US 2024/0060949 A1
(43) Pub. Date: Feb. 22, 2024

(54) BIOSYSTEMS TO DETECT CHEMICAL CONTAMINANTS

(71) Applicant: FUNDACION AZTI/AZTI FUNDAZIOA, Sukarrieto (ES)

(72) Inventors: Alejandro BARRANCO IBARBIA, Getxo (ES); Amaia EREÑO ARTABE, Amorebieta (ES); Hugo Flávio CUNHA DA SILVA, Bilbao (ES)

(21) Appl. No.: 18/269,781

(22) PCT Filed: Apr. 27, 2021
(86) PCT No.: PCT/EP2021/061028

§ 371 Date: Jun. 27, 2023

(30) Foreign Application Priority Data
Dec. 30, 2020 (EP) 20383173.0

(51) Int. Cl. G01N 33/02

(52) U.S. Cl. CPC G01N 33/02; G01N 2500/02

(57) ABSTRACT

The present invention refers to the field of food safety and environmental control. In particular, it refers to a method for the detection of chemical contaminants based on cytochrome P450 enzymes, to a kit to carry out said method and to its use.

### FIELD OF THE INVENTION

The present invention refers to the field of food safety and environmental control. In particular, it refers to a method for the detection of chemical contaminants based on cytochrome P450 enzymes.

### BACKGROUND OF THE INVENTION

Many chemical contaminants can enter in the food chain and finally be present in foodstuff. Due to their persistence and toxic properties, they pose a risk to consumers and to the environment which make some of them be regulated (i.e. polycyclic aromatic hydrocarbons (PAH), polychlorinated biphenyls (PCB), polychlorinated dibenzo para dioxins (dioxins) and polychlorinated dibenzofurans (furans), toxins from biological origin, pesticides, etc). There is also another group of contaminants, called “emerging contaminants”, which are not regulated but are of great concern because residues of these substances are becoming frequently detected and they have potential toxic effects too, for example, certain marine toxins, brominated flame retardants (BFR) or perfluorinated compounds (PFC). Despite the nature of chemical contaminants, conventional methodologies for their detection are based on chromatographic techniques that allow separating mixtures into individual components and their identification by coupled detectors such as mass spectrometers. Many methodologies describe suitable protocols for the extraction and detection of chemical contaminants in different matrices (Hird et al, 2014).

Chromatographic analysis is the gold standard technique to control the presence of chemical contaminants in food and environmental samples. Although chromatographic analysis is recognized by the high precision, sensitivity, and reproducibility of results, it is also associated with increased times and costs per analysis, and with the need of laboratory facilities to run the procedures.

Biosensors, very popular in health diagnostics (e.g. glucose sensor), have become an interesting tool for food monitoring due to its ease of use and manufacturing, low cost and suitability for field analysis. Many of them are based on immunoassays which are highly specific, so they are useful for the detection of only one compound or, in some cases, of a small group of compounds with similar chemical structures. However, chemical contaminants can be extremely different constraining to use a lot of antibodies which could finally be unfeasible. As an alternative, the use of enzymes and the monitoring of their catalytic activity can help to detect a group of different analytes by different ways: the detection of the resulting compounds from the catalytic activity, this is the case of the glucose biosensor, or the inhibition of this activity. As a consequence, a signal proportional to target analytes concentration is observed.

A number of enzyme biosensors have been developed for chemical contaminants, mostly using acetylcholinesterase or similar enzymes, that are inhibited by the presence of organophosphate compounds used as pesticides or warfare nerve agents (Palchetti, 2016). Moreover, there are several commercial biosensors based on these enzymes: acetylcholinesterase detection kit (Biosensor S.R.L), AChE sensor (BVT Technologies, a.s.), CideLite (Charm Sciences Inc). Although they can detect several contaminants, all of them are from the same family, organophosphates or N-methylcarbamates. Therefore, there is a need for enzymes capable of detecting the presence of many different chemical contaminants.

In the case of marine toxins, mostly used rapid methods and all commercial kits are based on immunoassays except for a few receptor binding assays and enzyme inhibition assays, namely protein phosphatase 2A (PP2A) and phosphodiesterase (PDE) (McPartlin et al, 2016). Immunoassays are also reported for environmental contaminants such as PAHs, polybrominated diphenyl ethers (BDEs), or PCBs and dioxins (Meimaridou et al., 2011). Chemical compounds can affect certain metabolic pathways of living organisms where many families of enzymes are involved. One of them is the family of cytochrome P450 (CYP450) enzymes that catalyses many of detoxification reactions of xenobiotics. CYP450 enzymes participate in phase I metabolism of xenobiotics. These enzymes detoxify and/or bioactivate a vast number of xenobiotic chemicals and their activities can be easily monitored by different methods such as the colorimetric detection of a chromophore or the detection of the enzyme substrates by Liquid Chromatography Mass Spectroscopy (LC-MS) (Bell et al, 2008).

The inductive or inhibitory effect of some chemical molecules has been tested for in vivo assessment of CYP450 activity, however the results cannot be translated to in vitro assays, since different biochemical cascades occur in such complex biological systems. Moreover, in vivo processes are dynamic and occur in, usually, larger time-lines, also presenting a dependence on the organs involved in the molecules' metabolism (Stevison et al., 2019). Some examples can be found in the literature for the differences and complexity of effects in vitro and in vivo assays of CYP450 enzyme reactions. For example, in vitro studies of gemfibrozil show that this molecule is not a potent inhibitor of CYP2C8 activity. However, in vivo data, indicate that gemfibrozil is metabolized by UDP-glucuronosyltransferase to gemfibrozil-1-o-β-glucuronide, a metabolism-based potent inhibitor of CYP2C8, revealing that in vitro data could not fully appreciated the in vivo effect (Ogilvie et al., 2006). Differences were also found by Chen and co-workers, disclosing contrasting data from in vivo and in vitro propofol metabolism by hamster hepatic CYP450 enzymes (Chen et al., 1995).

Despite the high amount of information (in vivo and in vitro) available for drugs, the use of CYP450s for the assessment of chemical contaminants is mostly for in vivo assessment and research on in vitro studies is scarce.

Therefore, there is still a need in the state of the art of providing methods in which CYP450 enzymes are used for the in vitro detection of different groups of chemical compounds or contaminants. Surprisingly, the authors of the present invention have developed said methods which are rapid, easily applicable and can be applied to different matrices (food, environmental, biological, etc) and to synthetic solutions. Moreover, they are useful for the detection of multiple CYP450 inhibitor chemical contaminants in a sample.

### OBJECT OF THE INVENTION

A first aspect of the present invention refers to a method for the detection of a CYP450 enzyme inhibitor chemical compound in a sample comprising the following steps:
a) contacting the sample or an extract thereof with a CYP450 enzyme allowing the interaction between the chemical compound and the CYP450;
b) addition of a CYP450 enzyme's substrate and incubation at suitable conditions to allow a catalytic reaction, between the substrate and the CYP450, that generates a detectable signal;
c) detection and measurement of the detectable signal;
d) determination of the presence of the compound by comparing the measured detectable signal with a reference sample;
wherein the chemical compound is selected from the group consisting of PFC, BFRs, PCBs, dioxins, furans, PAHs, toxins from biological origin, metals, food additives, feed additives and combinations thereof, and wherein the chemical compound is not a drug.

A second aspect of the present invention refers to a kit for carrying out the method according to the first aspect of the invention comprising:
a) a CYP450 enzyme;
b) a substrate of said CYP450 enzyme, wherein upon a catalytic reaction between the CYP450 enzyme and the substrate a detectable signal is generated;
c) a buffer solution in a pH of from 6 to 9;
d) means to detect and measure the detectable signal generated by the catalytic reaction of the CYP450 enzyme and the substrate; and
e) optionally, a NADPH generating system.

A third aspect of the present invention refers to the use of a kit as defined in the second aspect of the invention for detecting a CYP450 inhibitor chemical compound.

A fourth aspect of the invention refers to the use of a CYP450 enzyme for the detection of a CYP450 inhibitor chemical compound in a sample, wherein the CYP450 inhibitor chemical compound is not a drug and it is selected from the group consisting of PFC, BFR, PCBs, polychlorinated dibenzo para dioxins (dioxins), polychlorinated dibenzofurans (furans), PAHs, toxins from biological origin, metals, food additives, feed additives and combinations thereof, wherein the CYP450 enzyme is selected from of CYP1, CYP2, CYP3, CYP4, CYP17, CYP21, CYP46, CYP51 and combinations thereof, and wherein the sample is water, a biological sample, a food or feed sample, an environmental sample, or an extract thereof.

Other objects, features, advantages and aspects of the present application will become apparent to those skilled in the art from the following description and appended claims.

### BRIEF DESCRIPTION OF THE FIGURES

FIG. 1 shows the Log concentration-inhibition curve registered in the electrochemical assay for CYP3A4 response in presence of 50 μM progesterone and hexabromocyclododecane (HBCD) in the range from 0 to 300 ppbs (Example 4).

### DETAILED DESCRIPTION OF THE INVENTION

As used herein, the singular forms “a,” “an” and “the” include their corresponding plural forms unless the context clearly indicates otherwise. Unless defined otherwise, all the technical and scientific terms used herein have the same meaning as commonly understood by a person skilled in the art to which this invention belongs. To facilitate understanding and clarify the meaning of specific terms in the context of the present invention, the following definitions and particular and preferred embodiments thereof, applicable to all the embodiments of the different aspects of the present invention, are provided:

In the context of the present invention, “chemical compounds” refers to chemicals from anthropogenic and/or natural origin, excluding drugs, whose presence is not intended in a sample and display contaminant, pollutant, and toxicant properties for environment and living organisms. In this definition are included those substances whose presence is considered foreign for a specific sample and/or are above a permitted concentration level.

Drugs refer to drugs of abuse and/or drugs used with therapeutic and pharmaceutical purposes (e.g. active ingredient). In a particular embodiment, the chemical compound of the invention is not a pesticide either. Preferably, the chemical compound is selected from the group consisting of PFC, BFRs, PCBs, dioxins, furans, PAHs, toxins from biological origin, metals, food additives, feed additives and combinations thereof.

In the context of the present invention, “toxins from biological origin” refers to a substance produced by living organisms, that depending on the amount and route of exposure, is harmful and present toxic effects for another organism. As indicated above, these toxins exclude drugs of abuse and/or drugs used with therapeutic and pharmaceutical purposes. In a particular embodiment, the toxin from biological origin is selected from the group consisting of mycotoxins, microbial toxins, marine toxins, and combinations thereof.

In a first aspect, the present invention refers to a method for the detection of a CYP450 enzyme inhibitor chemical compound in a sample comprising the following steps:
a) contacting the sample or an extract thereof with a CYP450 enzyme allowing the interaction between the chemical compound and the CYP450;
b) addition of a CYP450 enzyme's substrate and incubation at suitable conditions to allow a catalytic reaction, between the substrate and the CYP450, that generates a detectable signal;
c) detection and measurement of the detectable signal;
d) determination of the presence of the compound by comparing the measured detectable signal with a reference sample;
wherein the chemical compound is selected from the group consisting of PFC, BFRs, PCBs, dioxins, furans, PAHs, toxins from biological origin, metals, food additives, feed additives and combinations thereof, and wherein the chemical compound is not a drug.

The reference sample can be a sample without the chemical compound to be detected or with a known concentration of said compound.

In a particular embodiment of the first aspect of the invention, the chemical compound is neither a drug nor a pesticide.

In another particular embodiment according to any of the previous embodiments, in step b) the catalytic reaction is selected from the group consisting of O-dealkylation, O-debenzylation, N-demethylation, hydroxylation, oxidation and combinations thereof. Preferably the reaction is O-dealkylation and/or O-debenzylation.

In a particular embodiment of the invention according to any one of the embodiments of the first aspect of the invention, in step c) the detectable signal is detected and measured by optical techniques (such as fluorometry, colorimetry, spectroscopy and surface plasmon resonance), electrochemical techniques (such as impedance, potentiometry, amperometry, coulometry and field-effect transistors), magnetic techniques (such as paramagnetic techniques, magneto-optical properties based techniques), mass-based techniques (such as quartz crystal microbalance, surface acoustic wave sensors) or thermistor-based techniques (such as calorimetry). Preferably, by optical techniques such as fluorometry, and by electrochemical techniques such as voltammetry, amperometry and impedance. As mentioned above, “a” includes the corresponding plural, that is, when referring to “a substrate” the corresponding plural (substrates) is included. Thus, one substrate or a combination of substrates can be use. In a particular embodiment of the invention according to any one of the embodiments of the first aspect of the invention, the CYP450 enzyme's substrate is characterized in that upon the catalytic reaction with the CYP450 generates a detectable signal. More particularly, the substrate is a fluorogenic substrate, which is converted to a fluorescent product by the catalytic reaction with the CYP450 enzyme, detectable by fluorometric techniques. In another particular embodiment, the substrate is electroactive and it is converted to a product by the catalytic reaction with the CYP450 enzyme, detectable by electrochemical techniques.

In another particular embodiment of the invention according to any one of the embodiments of the first aspect of the invention, the substrate is non-electroactive but the product resultant from the catalytic reaction between the substrate and the CYP450 is electroactive. In another particular embodiment, the detectable signal is generated by an electron exchange between the CYP450 and an electrode surface, in presence of any substrate of the CYP450. In these both last embodiments, the signal is detectable by electrochemical techniques.

In a particular embodiment of the invention according to any one of the embodiments of the first aspect of the invention, the substrate is selected from the group consisting of fluorescein derivatives, coumarin derivatives, progesterone, testosterone, caffeine, nicotine, estradiol, clozapine and combinations thereof. All these are known by the skilled in the art and/or commercially available. Examples of fluorescein derivatives are dibenzylfluorescein (DBF), di(benzyloxymethyl)fluorescein (DBOMF), benzyloxy-methylfluorescein (BOMF), -3-O-methylfluorescein (OMF), monobenzylfluorescein (MBF) and diethoxyfluorescein (DEF). Examples of coumarin derivatives are 7-Ethoxymethoxy-3-cyanocoumarin (EOMCC), 7-benzyloxymethyloxy-3-cyanocoumarin (BOMCC), 7-p-methoxy-benzyloxy-4-trifluoro-coumarin (MOBFC), 3-[2-(N, N-diethyl-N-methylami no)ethyl]-7-methoxy-4-methylcoumarin (AMMO), 3-[2-(N, N-diethyl-N-methylamino)ethyl]-7-methoxy-4-trifluoromethylcoumarin (MeAMFC), 7-methoxy-3-cyanocoumarin (CMC), 7-methoxy-4-trifluoromethylcoumarin (MFC), 7-benzyloxy-4-trifluoromethylcoumarin (BFC), 7-Benzyloxy-3-cyanocoumarin (BCC) and 7-hydroxy-4-trifluoromethylcoumarin (HFC). Any of these examples or mixtures thereof can be used in the present invention. More particularly, in the present invention, the fluorescein derivative is selected from the group consisting of DBF and/or DBOMF, and the coumarin derivative is selected from EOMCC and/or BOMCC. Preferably, the substrate is selected from the group consisting of DBF, DBOMF, EOMCC, BOMCC, progesterone and combinations thereof; and even more preferably is selected from DBF, DBOMF, EOMCC, BOMCC and combinations thereof, which are fluorogenic substrates easily detectable and, as shown in the Examples, have an excellent sensitivity.

In a particular embodiment of the invention according to any one of the embodiments of the first aspect of the invention, step d) further comprises determining the content of the compound. The content of the compound can be determined by any means known by the skilled in the art. In a particular embodiment, the content is determined by comparing the measured detectable signal with a reference sample. Preferably, in order to determine the concentration of the compound, a calibration is carried out and the percentage of inhibition is calculated versus a reference value. Representing the percentage of inhibition versus the concentration, provides a calibration that allows to infer the concentration of the compound in the sample. Advantageously, this embodiment of the present invention provides a quantitative method for the detection of a CYP inhibitor in a sample.

In a particular embodiment of the invention according to any one of the embodiments of the first aspect of the invention, in step a) the CYP450 enzyme is selected from the group consisting of CYP1, CYP2, CYP3, CYP4, CYP17, CYP21, CYP46, CYP51 and combinations thereof (i.e. family 1, 2, 3, 4, 17, 21, 46, 51 CYP450 enzymes and combinations thereof). More particularly, the CYP450 enzyme is selected from the group consisting of subfamilies CYP1A, CYP1B, CYP2A, CYP2B, CYP2C, CYP2D, CYP2E, CYP2J, CYP3A, CYP4A, CYP4F, CYP17A, CYP21A, CYP46A, CYP51A and combinations thereof. In a preferred embodiment the CYP450 enzyme is selected from the group consisting of CYP1A1, CYP1A2, CYP1B1, CYP2B4, CYP2B6, CYP2B10, CYP2B11, CYP2C9, CYP2C19, CYP2D6, CYP2E1, CYP2J2, CYP3A4, CYP3A5, CYP4F2, CYP4F3, CYP17A1, CYP46A1 and combinations thereof. More preferably, it is selected from the group consisting of CYP1A1, CYP1A2, CYP2B6, CYP2B10, CYP2B11, CYP2C19, CYP3A4 and combinations thereof, and even more preferably CYP1A1, CYP1A2, CYP2B6, CYP2B10, CYP2C19, CYP3A4, and combinations thereof (e.g. CYP1A2 and CYP2C19). Advantageously, when these enzymes are used low limits of detections, below the maximum legally allowed (when said maximum is established), is achieved. Besides, multianalyte detection is possible.

The CYP450 enzyme can be provided as purified recombinant enzyme, expressed and isolated from different sources in form of baculosomes, bactosomes or microsomes, or as a whole cell expressing CYP450. The CYP450 enzymes can be from different species, for example from human and/or from animal equivalents, such as mouse, rat, pig, dog and/or monkey. All these options are known by the skilled in the art and/or commercially available. The suitable conditions of the incubation of step b) are also known or easily determinable by the skilled in the art, and when the product is commercially available are provided by the supplier. These suitable conditions include a NADPH generating system, when required. The NADPH generating system generates the coenzyme form required for the CYP450 reaction with the substrate to occur. NADPH generating systems are known by the skilled in the art and are commercially available (e.g. Vivid® regeneration system). In a particular embodiment the NADPH generating system can use one or more enzymes selected from the group consisting of glucose dehydrogenase, citrate dehydrogenase and other NADPH-dependent enzymes.

In a particular embodiment of the invention according to any one of the embodiments of the first aspect of the invention, the chemical compound is a PFC, preferably selected from the group consisting of carboxylic acids, sulfonic acids, fluorotelomer alcohols, and combinations thereof. More preferably, the PFC is selected from the group consisting of perfluorooctanoic acid (PFOA), perfluorooctane sulfonic acid (PFOS) and combinations thereof. Advantageously, even when the PFC was detected in an extract of a food sample (fish, in Example 6) the level of detection was of the ng/g order, and the matrix did not have any effect affecting the correct detection of the compound (that is, no matrix effect was observed).

In a particular embodiment of the invention according to any one of the embodiments of the first aspect of the invention, the chemical compound is a BFR, preferably selected from the group consisting of Hexabromocyclododecane (HBCD), Tetrabromobisphenol A (TBBPA), brominated diphenyl ethers (BDEs) and combinations thereof. Examples of BDEs are 2,4,4′-Tribromodiphenyl ether (BDE-28); 2,2′,4,4′,5-Pentabromodiphenyl ether (BDE-99); 2,2′,4,4′-Tetrabromodiphenyl ether (BDE-47); 2,2′,4,4′,6-Pentabromodiphenyl ether (BDE-100); 2,2′,4,4′,5,5′-Hexabromodiphenyl ether (BDE-153); 2,2′,4,4′,5,6′-Hexabromodiphenyl ether (BDE-154); and Decabromodiphenyl ether (BDE-209).

Surprisingly, as shown in Examples 1, 3 and 4, levels of detection of few ng/mL are achieved with the method of the present invention, independently of the method of detection used (optical, lateral flow, electrochemical, respectively).

In a particular embodiment of the invention according to any one of the embodiments of the first aspect of the invention, the chemical compound is a PCB compound, preferably a dioxin-like PCB and/or non-dioxin like PCB (ICES-6). In a particular embodiment of the invention according to any one of the embodiments of the first aspect of the invention, the chemical compound is a dioxin and/or a furan. Advantageously, with the method of the invention, the level of detection of these compounds is in the range or below the maximum levels established by the Commission Regulation (EC) No 1881/2006 (see Examples 2 and 7).

In a particular embodiment of the invention according to any one of the embodiments of the first aspect of the invention, the chemical compound is a PAH compound, preferably selected from the group consisting of benzo[a]pyrene (BaP), benzo[b]fluoranthene (BbF), benzo(k)fluoranthene (BkF), benzo[a]anthracene (BaA), chrysene (Chrys), Dibenzo(a,h)anthracene (DbahA), Benzo(g,h,i)perylene (BghiP), Indeno[1,2,3-cd]pyrene (IP), pyrene (P), fluoranthene (FL), fluorene (F), phenanthrene (PA), anthracene (A), acenaphthylene (AP), acenaphthene (AC), naphthalene (NA), their derivatives, and combinations thereof. More preferably, the PAH compound is selected from the group consisting of BaP, BbF, BkF, BaA, Chrys, F, PA, AP, and a combination thereof, and even more preferably the PAH compound is selected from the group consisting of BaP, BbF, BaA, Chrys and a combination thereof, for which, as shown in Example 1, the limit of detection with the method of the invention is of as little as 5-20 μg/L.

In a particular embodiment of the invention according to any one of the embodiments of the first aspect of the invention, the chemical compound is a toxin from biological origin, preferably selected from the group consisting of mycotoxins, microbial toxins, marine toxins, and combinations thereof. More preferably, the toxin of biological origin is selected from the group consisting of azaspiracids, yessotoxins, pectenotoxins, okadaic acid and its derivatives, domoic acid, cyclic imines (such as spirolides), palytoxins, tetrotodoxins, saxitoxins, aflatoxins, and combinations thereof. Even more preferably, the toxin is Aflatoxin (e.g. B1, B2, G1, G2 or a mixture thereof), Azaspiracid-1, Yessotoxin, 13,19-didesmethyl C spirolide, Tetrodotoxin, for which a limit of detection of few μg/L is achieved with the method of the invention (data not shown and Examples 5 and 8).

In a particular embodiment of the invention according to any one of the embodiments of the first aspect of the invention, the chemical compound is a metal, more particularly it is a heavy metal. Preferably, the heavy metal is selected from the group consisting of lead, cadmium, mercury, methylmercury, tin (inorganic), arsenic and combinations thereof, and even more preferably the metal is mercury and/or methylmercury. Advantageously, the limit of detection of said metals with the method of the invention is of at least 31.7 ng/L (see Example 5).

In a particular embodiment of the invention according to any one of the embodiments of the first aspect of the invention, the chemical compound is a food and/or feed additive, more particularly selected from those additives with a regulated maximum limit. Preferably, the food and/or feed additive is selected from the group consisting of nitrite (such as sodium nitrite), nitrate, benzoate (such as sodium benzoate), monosodium glutamate, calcium carbonate, quinoline yellow, Sunset Yellow, Ponceau 4R, erythorbic acid, sodium ferrocyanide and combinations thereof. More preferably the additive is monosodium glutamate, sodium nitrite, sodium benzoate or a combination thereof. Advantageously, the method of the invention achieves low levels of detection of these additives, in particular, from 2 to 4 mg/mL (see Example 5).

In a preferred embodiment of the invention according to any one of the embodiments of the first aspect of the invention, the chemical compound is a PFC, and the CYP450 enzyme is selected from the group consisting of CYP2B, preferably CYP2B6, CYP46A and a combination thereof. More preferably, the substrate is selected from the group consisting of fluorescein derivatives and coumarin derivatives, even more preferably DBF, DBOMF, EOMCC, BOMCC, and combinations thereof.

In a preferred embodiment of the invention according to any one of the embodiments of the first aspect of the invention, the chemical compound is a BFR, and the CYP450 enzyme is selected from the group consisting of CYP1A, CYP2B, CYP2C, CYP3A and combinations thereof, preferably CYP1A2, CYP2B6, CYP3A4 and combinations thereof. More preferably, the substrate is selected from the group consisting of fluorescein derivatives and coumarin derivatives (preferably DBF, DBOMF, EOMCC, BOMCC and combinations thereof), or progesterone, and combinations thereof.

In a preferred embodiment of the invention according to any one of the embodiments of the first aspect of the invention, the chemical compound is selected from the group consisting of a PCB, dioxin, furan, and combinations thereof, and the CYP450 enzyme is selected from the group consisting of CYP1A (preferably CYP1A1), CYP1B, CYP2B, CYP2C, and combinations thereof. More preferably, the substrate is selected from the group consisting of fluorescein derivatives and coumarin derivatives, even more preferably DBF, DBOMF, EOMCC, BOMCC, and combinations thereof.

In a preferred embodiment of the invention according to any one of the embodiments of the first aspect of the invention, the chemical compound is a PAH, the CYP450 enzyme is selected from the group consisting of CYP1A, CYP1B, CYP2B (preferably CYP2B6) and combinations thereof. More preferably, the substrate is selected from the group consisting of fluorescein derivatives and coumarin derivatives, even more preferably DBF, DBOMF, EOMCC, BOMCC, and combinations thereof.

In a preferred embodiment of the invention according to any one of the embodiments of the first aspect of the invention, the chemical compound is a toxin from biological origin the CYP450 enzyme is selected from the group consisting of CYP1A, CYP2B, CYP2C, CYP3A, CYP46A and combinations thereof, preferably CYP1A2, CYP2B10, CYP2C19, CYP3A4 and combinations thereof (e.g. CYP1A2 and CYP2C19). More preferably, the substrate is selected from the group consisting of fluorescein derivatives and coumarin derivatives, even more preferably DBF, DBOMF, EOMCC, BOMCC, and combinations thereof.

In a preferred embodiment of the invention according to any one of the embodiments of the first aspect of the invention, the chemical compound is a metal, the CYP450 enzyme is selected from the group consisting of CYP1A, CYP2B, CYP3A, and combinations thereof, preferably CYP2B10 and/or CYP3A4. More preferably, the substrate is selected from the group consisting of fluorescein derivatives and coumarin derivatives (preferably DBF, DBOMF, EOMCC and/or BOMCC) or progesterone, and combinations thereof.

In a preferred embodiment of the invention according to any one of the embodiments of the first aspect of the invention, the chemical compound is a food and/or feed additive, the CYP450 enzyme is selected from the group consisting of CYP2B, CYP3A, and combinations thereof, preferably CYP2B6, CYP2B10, CYP3A4 and combinations thereof. More preferably, the substrate is selected from the group consisting of fluorescein derivatives and coumarin derivatives, even more preferably DBF, DBOMF, EOMCC, BOMCC, and combinations thereof.

As shown in the Examples, the combinations defined in the previous paragraphs provide excellent detection results, with very low limits of detection, and the matrix did not have any effect affecting the correct detection of the compound (when a matrix is used).

In a particular embodiment of the invention according to any one of the embodiments of the first aspect of the invention, the sample is selected from the group consisting of water, a biological sample (such as sputum, saliva, whole blood, serum, plasma, urine, feces, ejaculate, a buccal or buccal-pharyngeal swab, pleural fluid, peritoneal fluid, pericardic fluid, cerebrospinal fluid, intra-articular fluid, bronchial aspirates, or bronchioalveolar lavages, preferably saliva, whole blood or urine), a food sample, a feed sample, and an environmental sample. Preferably, the sample is a food, feed or environmental sample.

In a preferred embodiment according to any one of the embodiments of the first aspect of the invention, in step a) an extract of a sample is used, more preferably said extract is obtained by solvent extraction followed by a solid-phase extraction clean-up and a final evaporation step prior to reconstitution of the extract in a buffer solution.

The particular and preferred embodiments of the method of the invention described for the first aspect of the invention (e.g. CYP450, substrate, chemical compound, their particular combinations, etc) are applicable to the rest of the aspects of the invention.

A second aspect of the present invention refers to a kit for carrying out the method according to any one of the embodiments of the first aspect of the invention, wherein the kit comprises:
a) a CYP450 enzyme;
b) a substrate of said CYP450 enzyme, wherein upon a catalytic reaction between the CYP450 enzyme and the substrate a detectable signal is generated;
c) a buffer solution at a pH of from 6 to 9;
d) means to detect and measure the detectable signal generated by the catalytic reaction of the CYP450 enzyme and the substrate; and
e) optionally, a NADPH generating system, and/or
f) optionally, instructions of use.

Thus, the kit of the invention is an enzymatic assay of chemical compounds or contaminants capable of inhibiting CYP450 enzymes. That is, a biosystem for detecting chemical compounds or contaminants capable of inhibiting CYP450 enzymes.

The particular and preferred embodiments described in the first aspect of the invention are applicable to the second aspect of the invention (e.g. CYP450, substrate, chemical compound, their particular combinations, etc).

The CYP450 enzyme can be provided in the form of purified recombinant enzyme, particularly expressed and isolated from different sources in form of baculosomes, bactosomes or microsomes, or as a whole cell expressing CYP450 (as indicated for the method of the first aspect of the invention).

NADPH generating systems are known by the skilled in the art and can be commercially available (e.g. Vivid® regeneration system). The NADPH generating system is provided to generate the coenzyme form required for the CYP450 reaction with the substrate to occur. In a particular embodiment the NADPH generating system can use one or more enzymes selected from the group consisting of glucose dehydrogenase, citrate dehydrogenase and other NADPH-dependent enzymes.

For example, in a particular embodiment of the invention according to any one of the embodiments of the second aspect of the invention, the catalytic reaction is selected from the group consisting of O-dealkylation, O-debenzylation, N-demethylation, hydroxylation, oxidation, and combinations thereof, preferably O-dealkylation and/or O-debenzylation. For example, in a particular embodiment of the invention according to any one of the embodiments of the second aspect of the invention, the means to detect the detectable signal are selected from the group consisting of optical means, electrochemical means, magnetic means, mass-based means and thermistor-based means. In a particular embodiment, the kit comprises a NADPH generating system when the means for detection are any of the foregoing but electrochemical means.

For example, in a particular embodiment of the invention according to any one of the embodiments of the second aspect of the invention, the substrate is selected from the group consisting of a fluorescein derivative and a coumarin derivative (preferably DBF, DBOMF, EOMCC and/or BOMCC), progesterone, testosterone, caffeine, nicotine, estradiol and clozapine, and combinations thereof.

For example, in a particular embodiment of the invention according to any one of the embodiments of the second aspect of the invention, the CYP450 enzyme is selected from the group consisting of CYP1, CYP2, CYP3, CYP4, CYP17, CYP21, CYP46, CYP51 and combinations thereof. Preferably, the CYP450 enzyme is selected preferably from the group consisting of CYP1A, CYP1B, CYP2A, CYP2B, CYP2C, CYP2D, CYP2E, CYP2J, CYP3A, CYP4A, CYP4F, CYP17A, CYP21A, CYP46A, CYP51A and combinations thereof. And more preferably, the CYP450 enzyme is selected from the group consisting of CYP1A1, CYP1A2, CYP1B1, CYP2B4, CYP2B6, CYP2B10, CYP2B11, CYP2C9, CYP2C19, CYP2D6, CYP2E1, CYP2J2, CYP3A4, CYP3A5, CYP4F2, CYP4F3, CYP17A1, CYP46A1 and combinations thereof.

As explained above, the use of these particular combinations provides excellent detection results, with very low limits of detection, and the matrix did not have any effect affecting the correct detection of the compound (when a matrix is used).

A third aspect of the present invention refers to the use of a kit or biosystem as defined in any of the embodiments of the second aspect of the invention for detecting a CYP450 inhibitor chemical compound in a sample. More particularly, it refers to the use of a kit as defined in any of the embodiments of the second aspect of the invention for carrying out any one of the methods of the first aspect of the invention. The particular and preferred embodiments described in the first aspect of the invention are applicable to the third aspect of the invention (e.g. CYP450, substrate, chemical compound, their particular combinations, etc).

A fourth aspect of the present invention refers to a use of a CYP450 enzyme for the detection of a CYP450 inhibitor chemical compound in a sample, wherein the CYP450 inhibitor chemical compound is not a drug and it is selected from the group consisting of PFC, BFR, PCBs, polychlorinated dibenzo para dioxins (dioxins), polychlorinated dibenzofurans (furans), PAHs, toxins from biological origin, metals, food additives, feed additives and combinations thereof, wherein the CYP450 enzyme is selected from of CYP1, CYP2, CYP3, CYP4, CYP17, CYP21, CYP46, CYP51 and combinations thereof, and wherein the sample is water, a biological sample, a food or feed sample, an environmental sample, or an extract thereof. The particular and preferred embodiments described in the first aspect of the invention are applicable to the fourth aspect of the invention (e.g. CYP450 enzyme, chemical compound, their particular combinations, etc).

### REFERENCES

Bell, L., Bickford, S., Nguyen, P. H., Wang, J., He, T., Zhang, B., Friche, Y., Zimmerlin, L., Urban, L. & Bojanic, D. (2008). Evaluation of fluorescence—and mass spectrometry—based CYP inhibition assays for use in drug discovery. Journal of biomolecular screening, 13(5), 343-353.
Chen, T. L., Wang, H. J., Huang, C. H., Liu, C. C., Ueng, T. H. (1995). Difference between in vivo and in vitro effects of propofol on defluorination and metabolic activities of hamster hepatic cytochrome P450-dependent mono-oxygenases. Br. J. Anaesth. 75, 462-466.
Hird, S. J., Lau, B. P. Y., Schuhmacher, R., & Krska, R. (2014). Liquid chromatography-mass spectrometry for the determination of chemical contaminants in food. TrAC—Trends Anal. Chem. 59, 59-72.
McPartlin, D. A., Lochhead, M. J., Connell, L. B., Doucette, G. J., & O'Kennedy, R. J. (2016). Use of biosensors for the detection of marine toxins. Essays in biochemistry, 60(1), 49-58.
Meimaridou, A., Kalachova K., Shelver W. L., Franek M., Pulkrabova J., Haasnoot W., and Nielen M. W. F. (2011). Multiplex Screening of Persistent Organic Pollutants in Fish Using Spectrally Encoded Microspheres. Analytical Chemistry, 83, 22, 8696-8702.
Ogilvie, B. W., Zhang, D., Li, W., Rodrigues, A. D., Gipson, A. E., Holsapple, J., Toren, P. & Parkinson, A. (2006). Glucuronidation converts gemfibrozil to a potent, metabolism-dependent inhibitor of CYP2C8: Implications for drug-drug interactions. Drug Metab. Dispos. 34, 191-197.
Palchetti, I. (2016). Emerging Biosensor for Pesticide Detection. In Biosensors for Security and Bioterrorism Applications (pp. 431-442). Springer, Cham.
Stevison, F., Kosaka, M., Kenny, J. R., Wong, S., Hogarth, C., Amory, J. K., Isoherranen, N. (2019). Does In Vitro Cytochrome P450 Downregulation Translate to In Vivo Drug-Drug Interactions? Preclinical and Clinical Studies With 13-cis-Retinoic Acid. Clin. Transl. Sci. 12, 350-360.

### EXAMPLES

Specific embodiments of the invention that serve to illustrate the invention without limiting the scope thereof are described in detail below.

### Example 1—Detection of PAHs and BFRs by a CYP Inhibition Assay

The bioassay was performed in a 96-well plate. 50 μL of a solution containing PAHs or BFRs is added to wells in triplicate. Enzyme mixture was prepared following the protocols provided by the manufacturer (Thermofisher Scientific) and consisted of CYP1A2, CYP2B6, and CYP3A4 Baculosomes and Vivid® Reagent System (333 mM Glucose-6-phosphate and 30 U/mL Glucose-6-phosphate dehydrogenase in 100 mM potassium phosphate, pH=8.0) in working buffer. After the preparation, 50 μL of enzyme mixture were added to each plate. After incubating at room temperature 10 μL of substrate mixture (5 μM of substrate (EOMCC for CYP1A2, BOMCC for CYP2B6 or DBF for CYP3A4) and 30 μM Vivid® NADP⁺ diluted in working buffer) was added. The plate was incubated 30 minutes at 25° C. and when time passed, fluorescence signal was measured with a fluorometric reader, 410/490 nm and 460/520 nm were the excitation and emission wavelengths, respectively. A blank sample, buffer solution, was measured at the same time and the reduction of the signal, inhibition of enzyme activity, was calculated and associated with the presence of contaminants.

The sensitivity of this CYP assay was evaluated and limits of detection were estimated as the concentration providing 20% inhibition (IC20), which is also the limit used in ELISA assays.

Table 1 show the results obtained for PAHs, while Tables 2 and 3 show the results for different BFRs.

TABLE 1
Sensitivity of the methodology for detection of relevant
PAHs with CYP2B6.

| PAHs | Limit of detection μg/L |
|---|---|
| benzo(a)pyrene (BaP) | 15 |
| benz(a)anthracene (BaA) | 5 |
| chrysene (Chrys) | 10 |
| benzo(b)fluoranthene (BbF) | 20 |

As shown in Table 1, the sensitivity of the assay is in the range of few μg/L that makes it suitable for the analysis of PAHs in different matrices, such as food or environmental samples.

TABLE 2
Sensitivity of the methodology for detection of relevant
Polybrominated Diphenyl Ethers (PBDEs) with CYP3A4

| PBDEs | Limits of detection (ng/ml) |
|---|---|
| BDE-28 | 100 |
| BDE-47 | 5 |
| BDE-99 | 100 |
| BDE-100 | 5 |
| BDE-153 | 5 |
| BDE-154 | 5 |
| BDE-209 | 10 |

TABLE 3
Sensitivity of the methodology for detection of
relevant BFRs with CYP1A2.

| BFRs | Limits of detection (ng/ml) |
|---|---|
| HBCD | 50 |
| TBBPA | 50 |

As shown in Tables 2 and 3, the assay of the invention has a very good sensitivity for BFRs, in particular equal or higher than 5 ng/mL for BDEs and equal or higher than 50 ng/mL for HBCD and TBBPA.

### Example 2—Detecting PCBs and Dioxins by CYP450s Inhibition Assay

This inhibition assay was performed in a 96-well plate by adding 50 μL of PCBs and dioxins standards (single or mixtures) to wells in duplicate. Enzyme mixture consisted of 2 pmol/mL CYP1A1 in the assay buffer: 50 mM potassium phosphate, pH=7.4, containing 333 mM Glucose-6-phosphate and 30 U/mL Glucose-6-phosphate dehydrogenase. After preparation, 50 μL of enzyme mixture were added to each well and incubated for 10 minutes at room temperature. After incubation, 10 μL of a substrate mixture containing 30 μM NADP+ and 3 μM EOMCC in the phosphate buffer was added to the wells. The plate was incubated for 30 minutes at 25° C., and the fluorescence was monitored using 410/490 nm as excitation and emission wavelengths, respectively. A blank sample containing buffer solution was measured in same conditions and used to calculate the respective inhibitions of enzyme activity, that was associated with the presence of PCBs and dioxins.

The sensitivity of this CYP assay was evaluated and limits of detection were estimated as the concentration providing 20% inhibition (see Table 4).

TABLE 4
Sensitivity of the CYP assay for the different groups of PCBs and dioxins.

| Contaminants | Limits of detection |
|---|---|
| Dioxins | 1.8 pg TEQ/mL |
| Non-ortho PCBs | 7.3 pg TEQ/mL |
| Mono-ortho PCBs | 8.8 pg TEQ/mL |
| ICES-6 | 2.8 ng/ml |

The EU establishes the maximum level for these compounds in different foodstuff. In particular, it establishes:
0.3-3.5 μg TEQ/g for sum of dioxins,
1.25-20 μg TEQ/g for sum of dioxins and dioxin-like PCBs, and
3-300 ng/g for sum of PCB28, PCB52, PCB101, PCB138, PCB153 and PCB180 (ICES-6).

As shown in Table 4, all values were in the range or below the maximum levels established in EU (Commission Regulation (EC) No 1881/2006) for these compounds in different foodstuffs.

### Example 3—Detecting Brominated Contaminants by a CYP3A4 Inhibition Lateral Flow Assay

To demonstrate the applicability of lateral flow systems to detect brominated molecules, the inhibition response of CYP3A4 was evaluated for the presence of two contaminants BDE-100 and HBCD. The metabolism of two different fluorescent substrates was used to determine the percent of inhibition of CYP3A4 as response to the contaminant concentrations. The enzymatic reactions were carried out in 96-well plates adding 80 μL of potassium phosphate buffer (100 mM, pH 8.0) or 100 ng/mL of standard solutions in duplicates. Both brominated compounds were prepared in potassium phosphate buffer (100 mM, pH 8.0) and tested as inhibitors of CYP3A4 activity. The enzyme mixture was prepared following the protocols provided by the manufacturer (Thermofisher Scientific) and consisted of CYP3A4 Baculosomes and Vivid® Reagent System (333 mM Glucose-6-phosphate and 30 U/mL Glucose-6-phosphate dehydrogenase in 100 mM potassium phosphate, pH=8.0) in working buffer. Subsequently, 10 μL of enzyme mixture were added to each well, homogenised and allowed to pre-incubate during 10 minutes at room temperature. Two different substrate mixtures were used in combination with brominated molecules. To evaluate the inhibition promoted by BDE-100, 10 μL of a substrate mixture containing 2 μM of DBF and 30 μM Vivid® NADP+ in potassium phosphate buffer (100 mM, pH8.0), were added to the appropriated wells. The inhibitory effect of HBCD concentration was tested for a substrate mixture, containing 2 μM of DBOMF and 30 μM Vivid® NADP+ prepared in potassium phosphate buffer (100 mM, pH8.0), from which 10 μL were added to the appropriated wells. After the addition of the respective substrate probes, the plate was newly incubated at 25° C. for 60 minutes. The resulting reaction broths were diluted 10 and 20 times in buffer, for respectively DBF and BDE-100, and for DBOMF and HBCD reactions. Finally, 1.5 μL of diluted broths were pipetted as a line onto nitrocellulose membranes and the fluorometric measurements carried out at a lateral flow reader (ESEQuant LR3, QIAGEN) with a fluorometric channel of 470 nm and 520 nm as excitation and emission wavelengths, respectively. A blank sample, buffer solution, was measured at the same time and the reduction of the signal, inhibition of enzyme activity, was calculated and associated with the presence of each BDE, as shown in Table 5.

TABLE 5
Inhibition percentage (I (%)) calculated for CYP3A4 reactions in
presence of 100 ng/ml of BDE-100; 50 and 100 ng/ml of HBCD.

| Media composition | I (%) |
|---|---|
| 2 μM DBF | 0 |
| 2 μM DBF + 100 ng/ml BDE-100 | 55.4 |
| 2 μM DBOMF | 0 |
| 2 μM DBOMF + 50 ng/mL HBCD | 69.8 |
| 2 μM DBOMF + 100 ng/mL HBCD | 84.3 |

As seen in the table, the membrane used in this example proved to be a good platform to monitor CYP reactions and therefore, detect the presence of contaminants capable of inhibiting CYP reactions in an easy, sensitive and rapid way.

Considering the signal intensity developed for different levels of the contaminant molecules, the concentration providing 20% of inhibition was selected as limit of detection, estimated to be 36 ng/mL and 14 ng/mL for BDE-100 and HBCD, respectively.

### Example 4—Detecting HBCD by a CYP3A4 Inhibition Electrochemical Assay

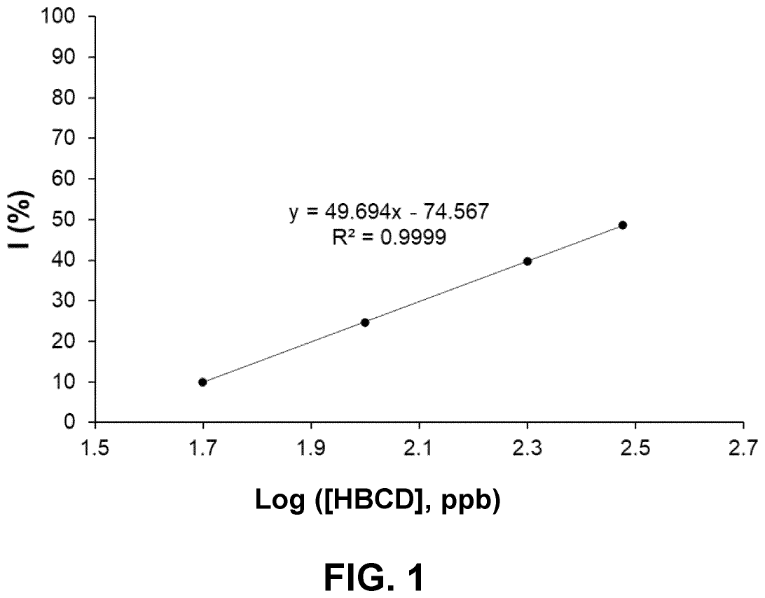

To demonstrate the applicability of electrochemical transducers to detect brominated molecules, the voltammetric signals from the inhibition response of CYP3A4 were evaluated in presence of HBCD. The working electrodes (WE) of screen-printed carbon electrodes (SPCE) (C110, from Methrom-Dropsens), were modified with 3 μL suspension of 1 mg/mL of single-walled carbon nanotubes. After chloroform evaporation, 5 μL of a 0.08 nmol/mL solution of CYP3A4 (recombinant, expressed in E. Coli from Sigma-Aldrich) was deposited onto the modified WE surface and incubated overnight at 4° C. 100 mM of potassium phosphate buffer saline (PBS) at pH 8.0, containing 50 mM of potassium chloride, was used to prepare the standards, as well as a supporting electrolyte for the electrochemical measurements. Aliquots (60 μL) of 50 μM progesterone solutions containing 0, 50, 100, 200 and 300 ng/mL of HBCD, were placed onto the working area of the SPCE by drop-casting, incubated for 2 minutes at room temperature and the electrochemical measurements performed. For each sample, one cyclic voltammetric scan was carried out, sweeping the potential from −0.75 V to 0.30 V at 0.02 V/s. The peak height of the cathodic peaks generated near to −0.55 V was registered and HBCD presence, calculated using as reference the peak of the blank assay (buffer solution). The electrochemical signal registered at CYP3A4-based biosensor, respond to the concentration of HBCD, allowing to establish the I (%) values for different concentrations of this compound (FIG. 1). Limit of detection was estimated to be 42.8 ng/mL for HBCD.

### Example 5—Detecting Mycotoxins, Metals, and Food and Feed Additives by a CYP450s Inhibition Assay

The bioassay was performed in a 96-well plate by adding 80 μL of mycotoxins (aflatoxins mixes), metals (mercury), and food and feed additives (sodium nitrite, sodium benzoate, monosodium glutamate) standards to wells in duplicate. Different enzyme mixtures were prepared depending on the contaminant and following the protocols provided by the manufacturers. CYP2B6 and CYP3A4 CYP450 Vivid® baculosomes (Thermofisher Scientific), and 0.25 pmol/mL of CYP2B10, from Cypex®, were mixed with Vivid® Regeneration System (333 mM Glucose-6-phosphate and 30 U/mL Glucose-6-phosphate dehydrogenase) in 50 mM potassium phosphate, pH=7.4, with exception of CYP3A4 that used 100 mM potassium phosphate, pH 8.0. After, 10 μL of each enzyme mixture were added to each plate and incubated for 10 minutes at room temperature. After this first incubation, 10 μL of substrate mixtures containing, 30 μM Vivid® NADP⁺ and 3 μM of BOMCC for CYP2B6 and CYP2B10, and 2 μM DBF for CYP3A4, were prepared in the respective buffers and added to the previous reactions. The plate was incubated for 30 minutes at 25° C., registering the fluorescence signals at 410/490 nm and 460/520 nm, which were the excitation and emission wavelengths, respectively. A blank sample containing buffer solution was measured in same conditions and used to calculate the respective inhibitions of enzyme activity, that was associated with the presence of contaminants. The results are shown in Tables 6-9. The limits of detection were defined as the concentration of contaminant/molecule that induces 20% inhibition of CYP450s activities.

TABLE 6
Sensitivity of the methodology for detection of Aflatoxins and
mercury with CYP2B10 and CYP3A4.

| | Limits of detection | |
|---|---|---|
| Contaminants/Molecules | CYP2B10 | CYP3A4 |
| Aflatoxins mix (B1, B2, G1, G2) (ng/mL) | 4.4 | 21 |
| Mercury (ng/ml) | 31.7 | 61.9 |

TABLE 7
Sensitivity of the methodology for detection of sodium nitrite with CYP2B6.

| | Limits of detection |
|---|---|
| Contaminants/Molecules | CYP2B6 |
| Sodium Nitrite (mg/mL) | 2.3 |

TABLE 8
Sensitivity of the methodology for detection of sodium benzoate and
methylmercury with CYP2B10.

| | Limits of detection |
|---|---|
| Contaminants/Molecules | CYP2B10 |
| Sodium Benzoate (mg/mL) | 3.2 |
| Methylmercury (ng/ml) | 44.3 |

TABLE 9
Sensitivity of the methodology for detection of Monosodium
glutamate with CYP3A4.

| | Limits of detection |
|---|---|
| Contaminants/Molecules | CYP3A4 |
| Monosodium glutamate (mg/mL) | 3.6 |

The results from Tables 6-9, demonstrate that different CYP450s can be inhibited by concentrations of mycotoxins, metals and/or food and feed additives, allowing to relate the I (%) with the presence of such compounds in samples.

### Example 6—Detection of PFCs in Fish Samples

The present invention was applied to the detection of PFOS and PFOA in an extract of fish samples. Briefly, the extraction method of the fish sample consisted of a methanolic alkaline digestion and a clean-up with strong anion exchange cartridges (SAX). The final elution from the SAX cartridge was performed in the assay working buffer containing 40% methanol.

The bioassay was performed in a 96-well plate. 50 μL of diluted extract (to 10% methanol) was added to wells in triplicate. Enzyme mixture was prepared following the protocols provided by the manufacturer (Thermofisher Scientific) and consisted of CYP2B6 Baculosomes and Vivid® Reagent System in potassium phosphate buffer 50 mM. After the preparation, 50 μL of enzyme mixture were added to each plate. After incubating at room temperature 10 μL of substrate mixture (5 μM BOMCC and 30 μM NADP+ diluted in potassium phosphate buffer 50 mM) was added. The plate was incubated 30 minutes at 25° C. and when time passed, fluorescence signal was measured with a fluorometric reader, 410 nm and 460 nm were the excitation and emission wavelengths, respectively. A blank sample, buffer solution, was measured at the same time and the reduction of the signal, inhibition of enzyme activity, was calculated and associated with the presence of PFCs. No matrix (fish sample) effect was observed with this methodology and the limit of detection (concentration that provided 20% inhibition) was calculated as 500 ng/g.

### Example 7—Detecting Dioxins by CYP450s Inhibition Assay in Feed Samples

An extract of the feed sample was obtained following a QuEChERs extraction (AOAC official method 2007.01) and a clean up by solid phase extraction (SiliaFast FaPex® Cereals). The resulting extract was evaporated and reconstituted in buffer assay containing 2% MeOH. The inhibition assay was performed in a 96-well plate by adding 50 μL of the extract to wells in duplicate. Enzyme mixture consisted of 2 pmol/mL CYP1A1 in the assay buffer: 50 mM potassium phosphate, pH=7.4, containing 333 mM Glucose-6-phosphate and 30 U/mL Glucose-6-phosphate dehydrogenase. After preparation, 50 μL of enzyme mixture were added to each plate and incubated for 10 minutes at room temperature. After incubation, 10 μL of a substrate mixture containing 30 μM NADP+ and 3 μM EOMCC in the phosphate buffer was added to the wells. The plate was incubated for 30 minutes at 25° C., and the fluorescence was monitored using 410/490 nm as excitation and emission wavelengths, respectively. A blank sample containing buffer solution was measured in same conditions and used to calculate the respective inhibitions of enzyme activity, that was associated with the presence of dioxins.

This methodology has been applied to feeds with different composition and designed for feeding different animals (pigs, chicken, cows and sheep). Advantageously, in all cases a similar matrix effect was obtained, so different limits of detection do not have to be established for each type of sample, and the limit of detection was established at 1 μg TEQ/g.

### Example 8—Detection of Marine Toxins

The bioassay was performed in a 96-well plate. 80 μL of sample is added to wells in triplicate. Enzyme mixture was prepared following the protocols provided by the manufacturer (Thermofisher Scientific) and consisted of CYP1A2 and CYP2C19 baculosomes and Vivid® Reagent System (333 mM Glucose-6-phosphate and 30 U/mL Glucose-6-phosphate dehydrogenase in 100 mM potassium phosphate, pH=8.0) in working buffer. After the preparation, 10 μL of enzyme mixture were added to each plate. After incubating at room temperature 10 μL of substrate mixture (5 μM of substrate EOMCC and 30 μM Vivid® NADP⁺ diluted in working buffer) was added. The plate was incubated 30 minutes at 30° C. and when time passed, fluorescence signal was measured with a fluorometric reader, 410 nm and 460 nm were the excitation and emission wavelengths, respectively. A blank sample, buffer solution, was measured at the same time and the reduction of the signal, inhibition of enzyme activity, was calculated and associated with the presence of contaminants. Table 10 shows the sensitivity of the assay. The limit of detection was defined as the concentration of contaminant/molecule that induces 20% inhibition of CYP450s activities.

TABLE 10
Sensitivity of the CYP assay for the different marine toxins.

| Marine Toxin | Limit of detection (μg/L) |
|---|---|
| Azaspiracid-1 | 3 |
| Yessotoxin | 45 |
| 13,19-didesmethyl C spirolide | 10 |
| Saxitoxin | 300 |

These results show that different CYP450s can be inhibited by concentrations of marine toxins, allowing to relate the I (%) with the presence of such compounds in the samples.

1. A method for the detection of a cytochrome P450 (CYP450) enzyme inhibitor chemical compound in a sample comprising the following steps:
a) contacting the sample or an extract thereof with a CYP450 enzyme allowing the interaction between the chemical compound and the CYP450;
b) addition of a CYP450 enzyme's substrate and incubation at suitable conditions to allow a catalytic reaction between the substrate and the CYP450, that generates a detectable signal;
c) detection and measurement of the detectable signal;
d) determination of the presence of the compound by comparing the measured detectable signal with a reference sample;
wherein the chemical compound is selected from the group consisting of perfluorinated compounds (PFC), brominated flame retardants (BFRs), polychlorinated biphenyls (PCBs), polychlorinated dibenzo para dioxins (dioxins), polychlorinated dibenzofurans (furans), polycyclic aromatic hydrocarbons (PAHs), toxins from biological origin, metals, food additives, feed additives and combinations thereof, and wherein the chemical compound is not a drug.

2. The method according to claim 1, wherein the compound is not a pesticide.

3. The method according to claim 1 or 2, wherein in step b) the catalytic reaction is selected from the group consisting of O-dealkylation, O-debenzylation, N-demethylation, hydroxylation, oxidation, and combinations thereof.

4. The method according to any one of the preceding claims, wherein in step c) the detectable signal is detected and measured by optical techniques, electrochemical techniques, magnetic techniques, mass-based techniques, thermistor-based techniques, and combinations thereof.

5. The method according to any one of the preceding claims, wherein step d) further comprises determination of the content of the compound.

6. The method according to any one of the preceding claims, wherein the substrate is selected from the group consisting of fluorescein derivative, coumarin derivative, progesterone, testosterone, caffeine, nicotine, estradiol and clozapine and combinations thereof.

7. The method according to claim 6, wherein the fluorescein derivative is selected from the group consisting of dibenzylfluorescein (DBF) and/or di(benzyloxymethyl)fluorescein (DBOMF), and/or the coumarin derivative is selected from 7-Ethoxymethoxy-3-cyanocoumarin (EOMCC) and/or 7-benzyloxymethyloxy-3-cyanocoumarin (BOMCC).

8. The method according to any one of the preceding claims, wherein the CYP450 enzyme is selected from the group consisting of CYP1, CYP2, CYP3, CYP4, CYP17, CYP21, CYP46, CYP51 and combinations thereof.

9. The method according to claim 8, wherein the CYP450 enzyme is selected preferably from the group consisting of CYP1A, CYP1B, CYP2A, CYP2B, CYP2C, CYP2D, CYP2E, CYP2J, CYP3A, CYP4A, CYP4F, CYP17A, CYP21A, CYP46A, CYP51A and combinations thereof.

10. The method according to claim 8 or 9, wherein the CYP450 enzyme is selected from the group consisting of CYP1A1, CYP1A2, CYP1B1, CYP2B4, CYP2B6, CYP2B10, CYP2B11, CYP2C9, CYP2C19, CYP2D6, CYP2E1, CYP2J2, CYP3A4, CYP3A5, CYP4F2, CYP4F3, CYP17A1, CYP46A1 and combinations thereof.

11. The method according to any one of the preceding claims, wherein the chemical compound is a PFC selected from the group consisting of carboxylic acids, sulfonic acids, fluorotelomer alcohols, and combinations thereof.

12. The method according to claim 11, wherein the PFC is selected from the group consisting of perfluorooctanoic acid (PFOA), perfluorooctane sulfonic acid (PFOS), and combinations thereof.

13. The method according to any one of claims 1 to 10, wherein the chemical compound is a BFR selected from the group consisting of Hexabromocyclododecane (HBCD), Tetrabromobisphenol A (TBBPA), brominated diphenyl ethers (BDEs) and combinations thereof.

14. The method according to any one of claims 1 to 10, wherein the chemical compound is selected from the group consisting of dioxin, furan, PCB compound selected from the group consisting of dioxin-like PCBs and/or non-dioxin like PCBs (ICES-6), and combinations thereof.

15. The method according to any one of claims 1 to 10, wherein the chemical compound is a PAH compound selected from the group consisting of benzo[a]pyrene (BaP), benzo[b]fluoranthene (BbF), benzo(k)fluoranthene (BkF), benzo[a]anthracene (BaA), chrysene (Chrys), Dibenzo(a,h)anthracene (DbahA), Benzo(g,h,i)perylene (BghiP), Indeno[1,2,3-cd]pyrene (IP), pyrene (P), fluoranthene (FL), fluorene (F), phenanthrene (PA), anthracene (A), acenaphthylene (AP), acenaphthene (AC), naphthalene (NA), their derivatives, and combinations thereof.

16. The method according to claim 15, wherein the PAH compound is selected from the group consisting of BaP, BbF, BkF, BaA, Chrys, F, PA, AP, and combinations thereof.

17. The method according to any one of claims 1 to 10, wherein the chemical compound is a toxin from biological origin selected from the group consisting of mycotoxins, microbial toxins, marine toxins, and combinations thereof.

18. The method according to claim 17, wherein the toxin of biological origin is selected from the group consisting of azaspiracids, yessotoxins, pectenotoxins, okadaic acid and its derivatives, domoic acid, cyclic imines, palytoxins, tetrotodoxins, saxitoxins, aflatoxins, and combinations thereof.

19. The method according to any one of claims 1 to 10, wherein the chemical compound is a metal.

20. The method according to claim 19, wherein the metal is a heavy metal selected from the group consisting of lead, cadmium, mercury, methylmercury, tin (inorganic), arsenic and combinations thereof, preferably mercury or methylmercury.

21. The method according to any one of claims 1 to 10, wherein chemical compound is a food and/or feed additive selected from those additives with a regulated maximum limit, preferably selected from the group consisting of nitrite, nitrate, benzoate, monosodium glutamate, calcium carbonate, quinoline yellow, Sunset Yellow, Ponceau 4R, erythorbic acid, sodium ferrocyanide and combinations thereof, preferably the additive is monosodium glutamate, sodium nitrite, sodium benzoate or a combination thereof.

22. The method according to claim 11 or 12, wherein the CYP450 enzyme is selected from the group consisting of CYP2B, CYP46A and combinations thereof.

23. The method according to claim 22, wherein the substrate is a fluorescein derivative and/or a coumarin derivative.

24. The method according to claim 13, wherein the CYP450 enzyme is selected from the group consisting of CYP1A, CYP2B, CYP2C, CYP3A and combinations thereof.

25. The method according to claim 24, wherein the substrate is selected from the group consisting of fluorescein derivative, coumarin derivative, progesterone, and combinations thereof.

26. The method according to claim 14, wherein the CYP450 enzyme is selected from the group consisting of CYP1A, CYP1B, CYP2B, CYP2C and combinations thereof.

27. The method according to claim 26, wherein the substrate is a fluorescein derivative and/or a coumarin derivative.

28. The method according to claim 15 or 16, wherein the CYP450 enzyme is selected from the group consisting of CYP1A, CYP1B, CYP2B and combinations thereof.

29. The method according to claim 28, wherein the substrate is a fluorescein derivative and/or a coumarin derivative.

30. The method according to claim 17 or 18, wherein the CYP450 enzyme is selected from the group consisting of CYP1A, CYP2B, CYP2C, CYP3A, CYP46A and combinations thereof.

31. The method according to claim 30, wherein the substrate is a fluorescein derivative and/or a coumarin derivative.

32. The method according to claim 19 or 20, wherein the CYP450 enzyme is selected from the group consisting of CYP1A, CYP2B, CYP3A and combinations thereof.

33. The method according to claim 32, wherein the substrate is selected from the group consisting of fluorescein derivative, coumarin derivative, progesterone, and combinations thereof.

34. The method according to claim 21, wherein the CYP450 enzyme is selected from the group consisting of CYP2B, CYP3A and combinations thereof.

35. The method according to claim 35, wherein the substrate is a fluorescein derivative and/or coumarin derivative.

36. The method according to any one of the preceding claims, wherein the sample is selected from the group consisting of water, a biological sample, a food or feed sample, and an environmental sample.

37. The method according to claim 36, wherein the sample is a food, feed or environmental sample.

38. Kit for carrying out the method according to any of the preceding claims comprising:
a) a CYP450 enzyme;
b) a substrate of said CYP450 enzyme, wherein upon a catalytic reaction between the CYP450 enzyme and the substrate a detectable signal is generated;
c) a buffer solution in a pH of 6-9;
d) means to detect and measure the detectable signal generated by the catalytic reaction of the CYP450 enzyme and the substrate; and
e) optionally, a NADPH generating system.

39. The kit according to claim 38, wherein the catalytic reaction is selected from the group consisting of O-dealkylation, O-debenzylation, N-demethylation, hydroxylation, oxidation, and combinations thereof.

40. The kit according to claim 38 or 39, wherein the means to detect the detectable signal are selected from the group consisting of optical means, electrochemical means, magnetic means, mass-based means and thermistor-based means.

41. The kit according to any one of claims 38-40, wherein the substrate is selected from the group consisting of fluorescein and coumarin derivative, progesterone, testosterone, caffeine, nicotine, estradiol, clozapine, and combinations thereof.

42. The kit according to any one of claims 38-41, wherein the CYP450 enzyme is selected from the group consisting of CYP1, CYP2, CYP3, CYP4, CYP17, CYP21, CYP46, CYP51 and combinations thereof.

43. The kit according to claim 42, wherein the CYP450 enzyme is selected preferably from the subgroup consisting of CYP1A, CYP1B, CYP2A, CYP2B, CYP2C, CYP2D, CYP2E, CYP2J, CYP3A, CYP4A, CYP4F, CYP17A, CYP21A, CYP46A, CYP51A and combinations thereof.

44. The kit according to claim 43, wherein the CYP450 enzyme is selected from the group consisting of CYP1A1, CYP1A2, CYP1B1, CYP2B4, CYP2B6, CYP2B10, CYP2B11, CYP2C9, CYP2D6, CYP2E1, CYP2J2, CYP3A4, CYP3A5, CYP4F2, CYP4F3, CYP17A1, CYP46A1 and combinations thereof.

45. Use of a kit as defined in any one of claims 38-44 for detection of a CYP450 enzyme inhibitor chemical compound in a sample.

46. Use according to claim 45, for carrying out the method according to any one of claims 1-37.

47. Use of a CYP450 enzyme for the detection of a CYP450 enzyme inhibitor chemical compound in a sample, wherein the CYP450 inhibitor chemical compound is not a drug and it is selected from the group consisting of PFC, BFR, PCBs, dioxins, furans, PAHs, toxins from biological origin, metals, food additives, feed additives and combinations thereof, wherein the CYP450 enzyme is selected from of CYP1, CYP2, CYP3, CYP4, CYP17, CYP21, CYP46, CYP51 and combinations thereof, and wherein the sample is water, a biological sample, a food, sample, a feed sample, an environmental sample, or an extract thereof.
